(19) United States
(12) Patent Application Publication
(10) Pub. No.: US 2019/0031830 A1
(43) Pub. Date: Jan. 31, 2019

(54) HIGH SOLIDS CONTENT POLYETHERIMIDE AND COMPONENTS THEREOF IN AN ORGANIC SOLVENT, AND METHOD OF PREPARATION

(71) Applicant: SABIC GLOBAL TECHNOLOGIES B.V., BERGEN OP ZOOM (NL)

(72) Inventors: Thomas Link Guggenheim, Mount Vernon, IN (US); Hendrich Chiong, Cincinnati, OH (US); Darlene Hope Nance, Mount Vernon, IN (US); Bernabe Quevedo Sanchez, Cartagena (ES); Carmen Rocio Misiego Arpa, Cartagena (ES); Juan Justino Rodriguez Ordonez, Cartagena (ES); Javier Nieves Remacha, Cartagena (ES)

(21) Appl. No.: 16/073,505

(22) PCT Filed: Jan. 26, 2017
(86) PCT No.: PCT/US2017/015114

§ 371 Date: Jul. 27, 2018

(30) Foreign Application Priority Data
Jan. 29, 2016 (EP) 16382038.4

(51) Int. Cl. C08G 73/10; C07D 209/48; C08K 5/053

(52) U.S. Cl. CPC C08G 73/1092; C08K 5/053; C07D 209/48

(57) ABSTRACT

A method is disclosed for the manufacture of a bis(phthalimide), and polyetherimides derived therefrom, by reacting a phthalic anhydride having the formula with an organic diamine having the formula H₂N—R—NH₂, in a solvent, at a temperature from 140° C. to 220° C., and a pressure from 0 psig to 100 psig, in the presence or absence of a phase transfer catalyst, to form a bis(phthalimide) having the formula wherein a polyetherimide polymer is added either before or after the imidization reaction, to produce a bis(phthalimide) composition having a percent solids content of 18% to 30%, which bis(phthalimide) composition has a viscosity of less than 4000 cP at a shear rate of less than 30 sec−1 and temperaturefrom 140° C. to 180° C. as measured in a spindle viscometer.

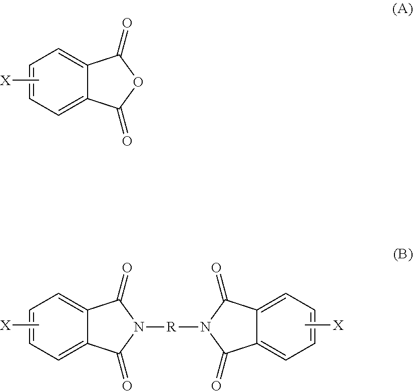

### BACKGROUND

This disclosure relates to a method for the manufacture of polyetherimides.

To meet the increased demand for polyetherimide, a new process has been developed, commonly referred to as the “displacement polymerization” process. Synthesis of polyetherimides via the displacement polymerization process includes imidization as described, for example, in U.S. Pat. No. 6,235,866), to produce a bisphthalimide substituted with a leaving group; synthesis of a salt of a dihydroxy aromatic compound, as described, for example, in U.S. Pat. No. 4,520,204; and polymerization by reacting the substituted bisphthalimide and the salt (“displacement polymerization”), as described, for example, in U.S. Pat. No. 6,265,521, followed by downstream activities.

In particular, imidization generally proceeds by reaction of 2 moles of a phthalic anhydride substituted with a leaving group with 1 mole of diamine in a reaction solvent, such as ortho-dichlorobenzene (ODCB) to provide a bis(phthalimide) substituted with two leaving groups. In a specific embodiment, the substituted phthalic anhydride is 4-chlorophthalic anhydride, the diamine is meta-phenylene diamine, and the bisphthalimide is a bis(chlorophthalimide) (ClPAMI). When 3-chlorophthalic anhydride (3-ClPA) and 4,4-diaminodiphenyl sulfone (DDS) are used, the product is 4,4′-bis(phenyl-3-chlorophthalimide)sulfone (DDS ClPAMI)). The bis(phthalimide) polymerizes with Bisphenol A disodium salt (BPANa₂) to provide the polyetherimide via chloro-displacement in the presence of a phase transfer catalyst, such as hexaethylguanidinium chloride (HEGCl). Utilization of HEGCl as a phase transfer catalyst at higher temperatures is described in U.S. Pat. No. 5,229,482.

The manufacture of polyetherimide via chloro-displacement requires the formation of ClPAMI as a slurry in a solvent, such as ortho-dichlorobenzene (ODCB). At a temperature at or near ODCB's boiling point and in the presence of an imidization catalyst such as hexaethylguanidinium chloride (HEGCl), the ClPAMI slurry can be made with very low levels of unreacted starting materials. However, ClPAMI in ODCB forms a thixotropic mixture. This property becomes more pronounced at concentrations higher than 20% solids and at temperatures lower than the boiling point of ODCB. A concentration higher than 20% solids can lead to operational issues such as sticking of the ClPAMI to the sides of the vessel, and product issues such as high residual unreacted ClPAMI in the resulting polyetherimide. However, setting the upper limit for dichloro-bisphthalimide concentration at less than 20% solids also limits the amount of polyetherimide that can be made in a batch or in a continuous process.

Accordingly, there is an ongoing need for a process for the manufacture of polyetherimides that does not suffer from these disadvantages.

### BRIEF DESCRIPTION

A method is disclosed for the manufacture of a polyetherimide composition, the method comprising imidizing a phthalic anhydride having the formula

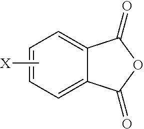

with an organic diamine having the formula H₂N—R—NH₂, in a solvent, at a temperature from 140° C. to 220° C., and a pressure from 0 psig to 100 psig, in the presence or absence of a phase transfer catalyst, to form a bis(phthalimide) having the formula

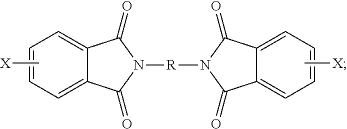

and polymerizing the bis(phthalimide) with an alkali metal salt of a dihydroxy aromatic compound having the formula

MO—Z—OM

via displacement to form the polyetherimide comprising structural units having the formula

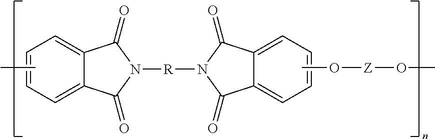

wherein in the foregoing formulas X is fluoro, chloro, bromo, iodo, nitro, or a combination comprising at least one of the foregoing; and R is a C₆₋₂₀ aromatic hydrocarbon group or a halogenated derivative thereof, a straight or branched chain C₂₋₂₀ alkylene group or a halogenated derivative thereof, a C₃₋₈ cycloalkylene group or halogenated derivative thereof, in particular a divalent group of one or more of the following formulas

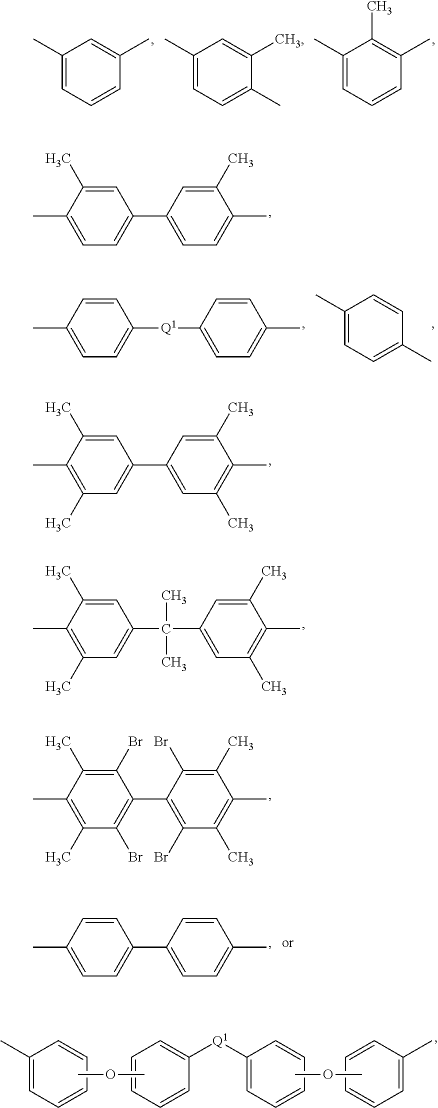

wherein Q¹ is —O—, —S—, —C(O)—, —SO₂—, —SO—, —C_{y}H_{2y}— wherein y is an integer from 1 to 5 or a halogenated derivative thereof, or —(C₆H₁₀)_{z}— wherein z is an integer from 1 to 4, or a combination comprising at least one of the foregoing; M is an alkali metal; Z is a divalent aromatic C₆₋₂₄ monocyclic or polycyclic moiety; and n is an integer greater than 1; and wherein a polyetherimide polymer is added either before or after the imidization reaction, to produce a bis(phthalimide) composition having a percent solids content of 18% to 30%, which bis(phthalimide) composition has a viscosity of less than 4000 cP at a shear rate of less than 30 sec⁻¹ and at a temperature from 140° C. to 180° C. as measured in a spindle viscometer.

A method is disclosed for the manufacture of a bis(phthalimide) comprising imidizing a phthalic anhydride having the formula

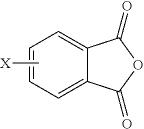

wherein X is a leaving group, preferably chlorine, with an organic diamine having the formula H₂N—R—NH₂, in a solvent, at a temperature from 140° C. to 220° C., and a pressure from 0 psig to 100 psig, in the presence or absence of a phase transfer catalyst, to form a bis(phthalimide) having the formula

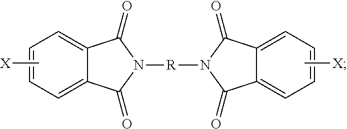

wherein a polyetherimide polymer is added either before or after the imidization reaction, to produce a bis(phthalimide) composition having a percent solids content of 18% to 30%, which bis(phthalimide) composition has a viscosity of less than 4000 cP at a shear rate of less than 30 sec⁻¹ and at a temperature from 140° C. to 180° C. as measured in a spindle viscometer.

A method is disclosed for reducing the viscosity of a slurry of 10 to 30% solids ClPAMI in ortho-dichlorobenzene, by adding a polyetherimide polymer in an amount from 0.5 to 5 weight percent, or from 1 to 3 weight percent, or from 1 to 2 weight percent, each based on the weight of the ClPAMI solids, to provide improved mixing properties with a reduction in the slurry viscosity of 20% to 60%, at temperatures of 140° C. to 220° C., over a range of shear rates of 50 sec⁻¹ to 5 sec⁻¹ as measured in a spindle viscometer.

A method is disclosed for producing ClPAMI at 30% solids in ortho-dichlorobenzene by adding a polyetherimide polymer in an amount from 0.5 to 5 weight percent, or from 1 to 3 weight percent, or from 1 to 2 weight percent, each based on the weight ClPAMI solids, to produce a 30% solids ClPAMI slurry in ortho-dichlorobenzene, having a viscosity of less than 4000 cP at a shear rate of less than 30 sec⁻¹ at 165° C. as measured in a spindle viscometer.

A bis(phthalimide) product is disclosed having a solids content of 30% and a viscosity of less than 4000 cP at a shear rate of less than 30 sec⁻¹ at 165° C. as measured in a spindle viscometer.

A method is disclosed for preparing a bisphenol A disodium slurry in ortho-dichlorobenzene comprising charging ortho-dichlorobenzene and a polyetherimide polymer to a reactor, maintaining the reactor temperature above 100° C., and then gradually adding aqueous bisphenol A disodium slurry to the reactor.

### BRIEF DESCRIPTION OF THE DRAWINGS

The following Figures are exemplary embodiments.

FIG. 1 is a graph of Shear rate vs. Viscosity for a ClPAMI slurry (17.3 wt. % solids based on weight of ODCB) according to the known process at 160° C., 170° C., and 180° C. (three runs each). The graph is presented on a logarithmic scale, which readily illustrates that the viscosity of the ClPAMI slurry at 160° C. is a multiple of the viscosity at 180° C.

FIG. 2 is a graph of Shear rate vs. Viscosity for ClPAMI slurries. The graph illustrates that for 20 weight percent solids at 165° C., a slurry containing 1% added polyetherimide polymer has a 40% lower viscosity than a ClPAMI slurry without the added polymer.

FIG. 3 is a graph of Shear rate vs. Viscosity, which shows that a ClPAMI slurry containing 2% polyetherimide polymer had a reduced viscosity compared against a ClPAMI slurry containing 1% polyetherimide polymer at 120° C.

FIG. 4 is a graph of Shear rate vs. Viscosity, which shows that a ClPAMI slurry containing 2% polyetherimide polymer had a reduced viscosity compared against a ClPAMI slurry containing 1% polyetherimide polymer at 140° C.

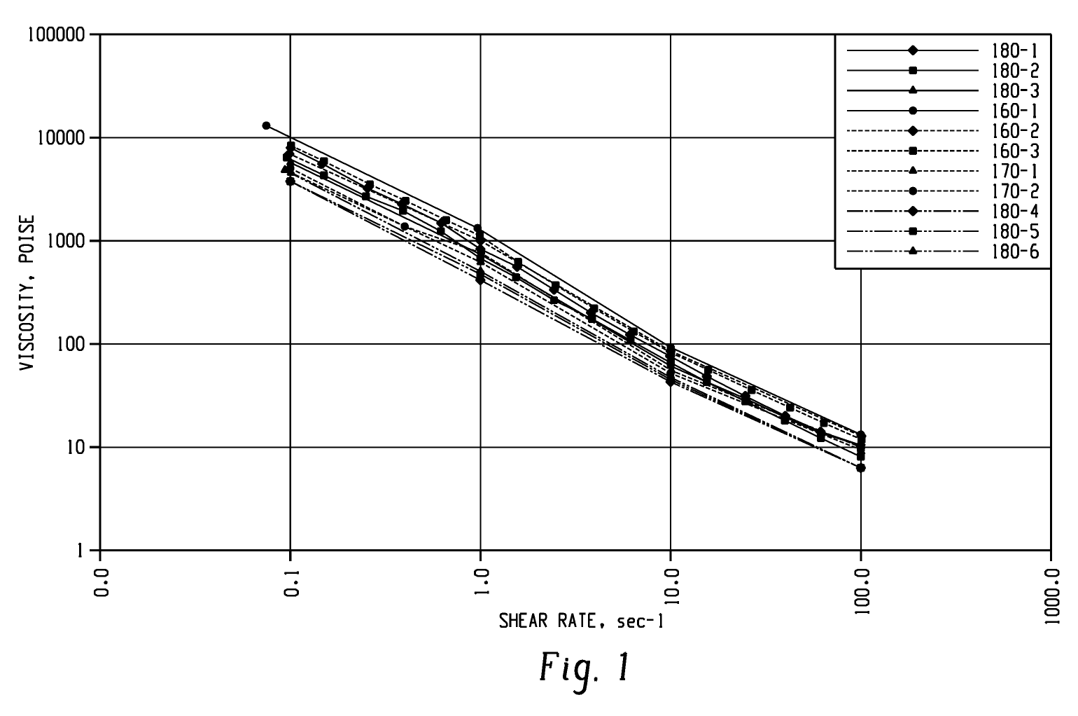

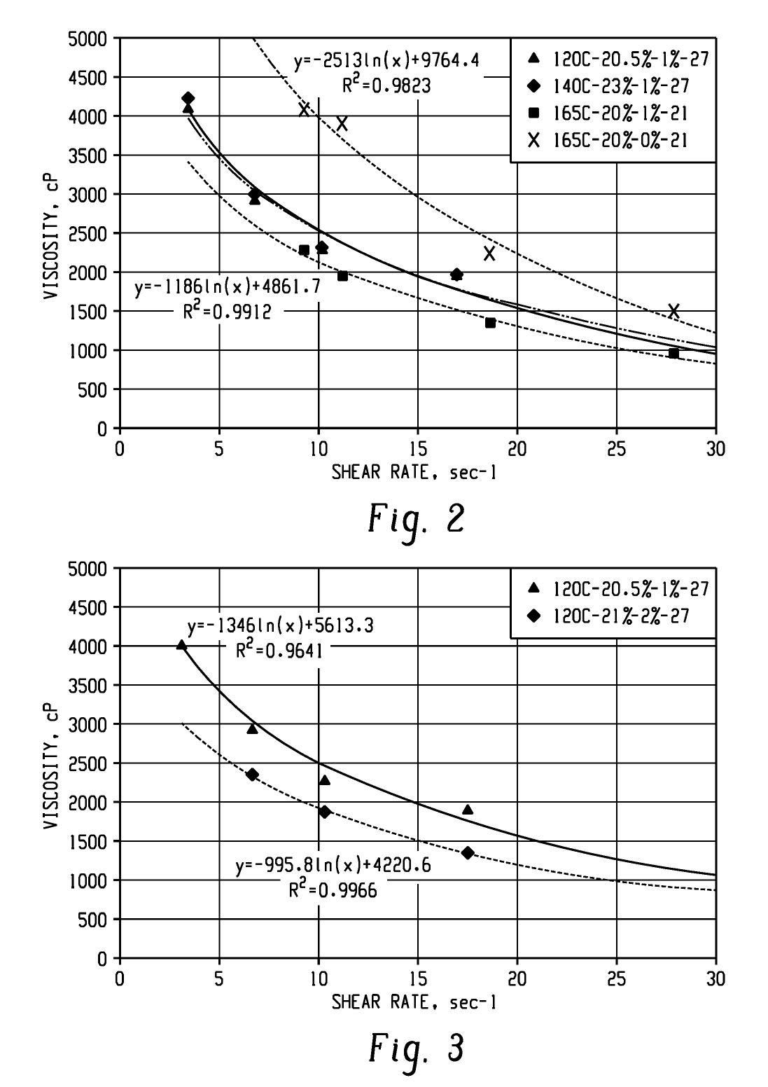

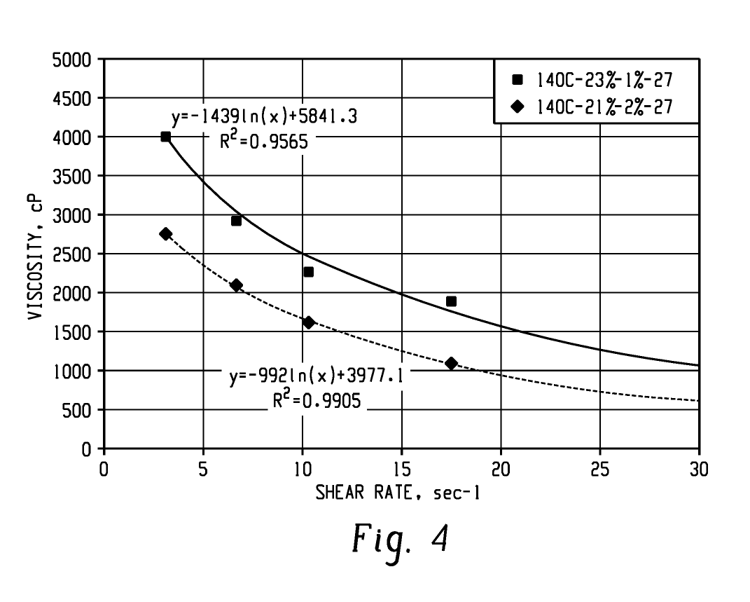

### DETAILED DESCRIPTION

It has been discovered that by adding a small amount of polymer, e.g., polyetherimide, either in a reaction mixture containing substituted anhydride and diamine prior to imidization, or to a bis(phthalimide) product, the viscosity of the resulting bis(phthalimide) product slurry was reduced at operating temperatures, allowing for very efficient mixing. Reduction of viscosity was observed whenever the polyetherimide was added to the bis(phthalimide) slurry (i.e., either at the beginning during, or at the end of the ClPAMI forming process). The reduction in viscosity produced easier stirring/mixing of ClPAMI slurry even at concentrations higher than 20% solids, and lowered the measured viscosity. It has also been found that reducing the viscosity of a slurry of 10 to 30% solids ClPAMI in ortho-dichlorobenzene can be accomplished by adding a polyetherimide polymer in an amount from 0.5 to 5 weight percent, or from 1 to 3 weight percent, or from 1 to 2 weight percent, each based on the weight of the ClPAMI solids, to provide improved mixing properties, with a reduction in the slurry viscosity of 20% to 60%, or 30% to 45% at temperatures of 140° C. to 220° C., or 150° C. to 190° C., or about 165° C., over ranges of shear rates of 50 sec⁻¹ to 5 sec⁻¹, or 28 sec⁻¹ to 8 sec⁻¹ as measured in a spindle viscometer.

The ClPAMI slurries can be produced at concentrations of 30% solids having a viscosity of less than 4000 cP at a shear rate of less than 30 sec⁻¹, or less than 15 sec⁻¹, or less than 10 sec⁻¹ as measured in a spindle viscometer.

The polyetherimide polymer can be added to the reaction mixture in various forms, such as polyetherimide polymer pellets, polyetherimide polymer pre-devitalization solution, and polyetherimide polymer oligomers. The decrease in viscosity correlates with the amount of polymer employed in the ClPAMI forming reaction.

In some embodiments, the amount of polyetherimide polymer to be added can be from 0.5 weight percent to 5 weight percent; from 1 weight percent to 3 weight percent; from 1 to 2 weight percent, all based upon total weight of reactants.

This process provides a number of advantages for the preparation of bis(phthalimide)s, in particular bis(halophthalimide)s such as ClPAMI, for example, eliminating the need to employ high temperature, high pressure (230° C./25 psig (pounds per square inch, gauge)) conditions in bis(phthalimide)synthesis. This also allows stoichiometry adjustments to be made at ambient pressure and at lower temperature, such as 180° C. Due to the lower viscosity of the bis(phthalimide) product, there is no longer a need to dilute from 25% to 20% solids after bis(phthalimide) synthesis and before aromatic dihydroxy salt addition, which allows an increase in polymer batch size.

Polyetherimides that can be manufactured using this process comprise more than 1, for example 10 to 1000, or 10 to 500, or 10 to 100 structural units of formula (1)

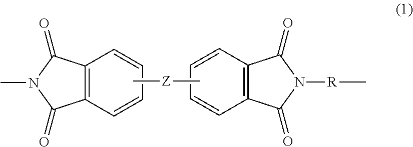

wherein each R is independently the same or different, and is a substituted or unsubstituted divalent organic group, such as a C₆₋₂₀ aromatic hydrocarbon group or a halogenated derivative thereof, a straight or branched chain C₂₋₂₀ alkylene group or a halogenated derivative thereof, a C₃₋₈ cycloalkylene group or halogenated derivative thereof, in particular a divalent group of one or more of the following formulas (2)

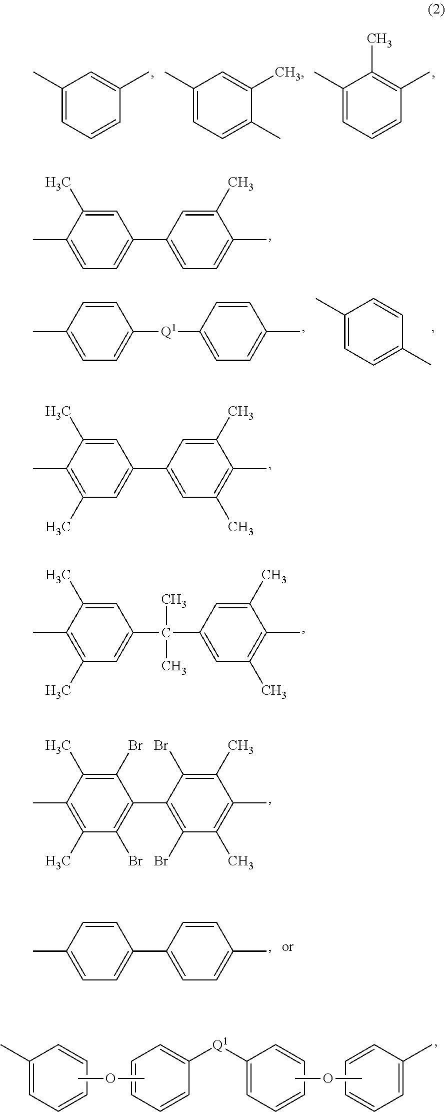

wherein Q¹ is —O—, —S—, —C(O)—, —SO₂—, —SO—, —P(R^{a})(═O)— wherein Ra is a C₁₋₈ alkyl or C₆₋₁₂ aryl, —C_{y}H_{2y}— wherein y is an integer from 1 to 5 or a halogenated derivative thereof (which includes perfluoroalkylene groups), or —(C₆H₁₀)_{z}— wherein z is an integer from 1 to 4. In some embodiments R is m-phenylene, p-phenylene, bis(4,4′-phenylene)sulfone, bis(3,4′-phenylene)sulfone, bis(3,3′-phenylene)sulfone, or a combination comprising at least one of the foregoing. In some embodiments, at least 10 mole percent or at least 50 mole percent of the R groups contain sulfone groups, and in other embodiments no R groups contain sulfone groups.

Further in formula (1), the divalent bonds of the —O— or the —O—Z—O— group are in the 3,3′, 3,4′, 4,3′, or the 4,4′ positions, and Z is an aromatic C₆₋₂₄ monocyclic or polycyclic moiety. Exemplary groups Z include groups of formula (3)

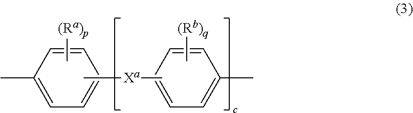

wherein R^{a} and R^{b} are each independently the same or different, and are a halogen atom or a monovalent C₁₋₆ alkyl group, for example; p and q are each independently integers of 0 to 4; c is 0 to 4; and X^{a} is a bridging group connecting the hydroxy-substituted aromatic groups, where the bridging group and the hydroxy substituent of each C6 arylene group are disposed ortho, meta, or para (specifically para) to each other on the C6 arylene group. The bridging group X^{a} can be a single bond, —O—, —S—, —S(O)—, —S(O)₂—, —C(O)—, or a C₁₋₁₈ organic bridging group. The C₁₋₁₈ organic bridging group can be cyclic or acyclic, aromatic or non-aromatic, and can further comprise heteroatoms such as halogens, oxygen, nitrogen, sulfur, silicon, or phosphorous. The C₁₋₁₈ organic group can be disposed such that the C₆ arylene groups connected thereto are each connected to a common alkylidene carbon or to different carbons of the C₁₋₁₈ organic bridging group. A specific example of a group Z is a divalent group of formula (3a)

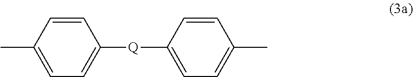

wherein Q is —O—, —S—, —C(O)—, —SO₂—, —SO—, P(R^{a})(═O)— wherein R^{a} is a C₁₋₈ alkyl or C₆₋₁₂ aryl, or —C_{y}H_{2y}— wherein y is an integer from 1 to 5 or a halogenated derivative thereof (including a perfluoroalkylene group). In a specific embodiment Z is a derived from bisphenol A, such that Q in formula (3a) is 2,2-isopropylidene.

In an embodiment in formula (1), the polyetherimide comprises more than 1, specifically 10 to 1,000, or more specifically, 10 to 50 structural units, and R is m-phenylene, p-phenylene, or a combination comprising at least one of the foregoing, and T is —O—Z—O— wherein Z is a divalent group of formula (3a). Alternatively, R is m-phenylene, p-phenylene, or a combination comprising at least one of the foregoing, and T is —O—Z—O wherein Z is a divalent group of formula (3a) and Q is 2,2-isopropylidene. In some embodiments, the polyetherimide can be a copolymer comprising additional structural polyetherimide units of formula (1) wherein at least 50 mole % (mol %) of the R groups are bis(3,4′-phenylene)sulfone, bis(3,3′-phenylene)sulfone, or a combination comprising at least one of the foregoing, and the remaining R groups are p-phenylene, m-phenylene or a combination comprising at least one of the foregoing; and Z is 2,2-(4-phenylene)isopropylidene, i.e., a bisphenol A moiety.

In some embodiments, the polyetherimide is a copolymer that optionally comprises additional structural imide units that are not polyetherimide units, for example imide units of formula (4)

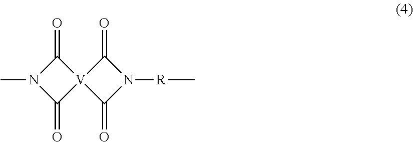

wherein R is as described in formula (1) and each V is the same or different, and is a substituted or unsubstituted C₆₋₂₀ aromatic hydrocarbon group, for example a tetravalent linker of the formulas

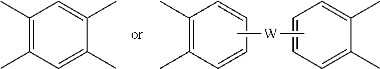

wherein W is a single bond, —S—, —C(O)—, —SO₂—, —SO—, or —C_{y}H_{2y}— wherein y is an integer from 1 to 5 or a halogenated derivative thereof (which includes perfluoroalkylene groups). These additional structural imide units preferably comprise less than 20 mol % of the total number of units, and more preferably can be present in amounts of 0 to 10 mol % of the total number of units, or 0 to 5 mol % of the total number of units, or 0 to 2 mole % of the total number of units. In some embodiments, no additional imide units are present in the polyetherimide.

The polyetherimides can have a melt index of 0.1 to 10 grams per minute (g/min), as measured by American Society for Testing Materials (ASTM) D1238 at 340 to 370° C., using a 6.7 kilogram (kg) weight. In some embodiments, the polyetherimide polymer has a weight average molecular weight (Mw) of 1,000 to 150,000 grams/mole (Dalton), as measured by gel permeation chromatography, using polystyrene standards. In some embodiments the polyetherimide has an Mw of 10,000 to 80,000 Daltons. Such polyetherimide polymers typically have an intrinsic viscosity greater than 0.2 deciliters per gram (dl/g), or, more specifically, 0.35 to 0.7 dl/g as measured in m-cresol at 25° C.

The polyetherimides are prepared by the so-called “displacement” polymerization method. In this method, a substituted phthalic anhydride of formula (7)

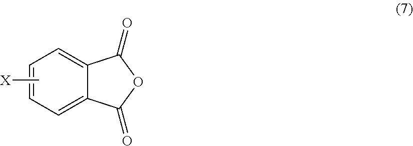

wherein X is a halogen or nitro, is reacted (imidized) with an organic diamine of the formula (8)

H₂N—R—NH₂ (8)

wherein R is as described in formula (1), to form a bis(phthalimide) of formula (9).

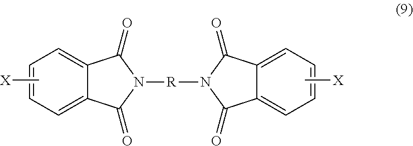

In some embodiments, X is a halogen, such as fluoro, chloro, bromo, or iodo, or nitro. In some embodiments, X is chloro. A combination of different halogens can be used.

Examples of organic diamines (8) include 1,4-butanediamine, 1,5-pentanediamine, 1,6-hexanediamine, methylated and polymethylated derivatives of the foregoing, heptamethylenediamine, octamethylenediamine, nonamethylenediamine, decamethylenediamine, 1,12-dodecanediamine, 1,18-octadecanediamine, 3-methylheptamethylenediamine, 4,4-dimethylheptamethylenediamine, 4-methylnonamethylenediamine, 5-methylnonamethylenediamine, 2,5-dimethylhexamethylenediamine, 2,5-dimethylheptamethylenediamine, 2,2-dimethylpropylenediamine, N-methyl-bis (3-aminopropyl) amine, 3-methoxyhexamethylenediamine, 1,2-bis(3-aminopropoxy) ethane, bis(3-aminopropyl) sulfide, 1,4-cyclohexanediamine, bis-(4-aminocyclohexyl) methane, m-phenylenediamine, p-phenylenediamine, 2,4-diaminotoluene, 2,6-diaminotoluene, m-xylylenediamine, p-xylylenediamine, 2-methyl-4,6-diethyl-1,3-phenylene-diamine, 5-methyl-4,6-diethyl-1,3-phenylene-diamine, benzidine, 3,3′-dimethylbenzidine, 3,3′-dimethoxybenzidine, 1,5-diaminonaphthalene, bis(4-aminophenyl) methane, bis(2-chloro-4-amino-3,5-diethylphenyl) methane, bis(4-aminophenyl) propane, 2,4-bis(p-amino-t-butyl) toluene, bis(p-amino-t-butylphenyl) ether, bis(p-methyl-o-aminophenyl) benzene, bis(p-methyl-o-aminopentyl) benzene, 1,3-diamino-4-isopropylbenzene, bis(4-aminophenyl) sulfide, bis-(4-aminophenyl) sulfone (also known as 4,4′-diaminodiphenyl sulfone (DDS)), and bis(4-aminophenyl) ether. Any regioisomer of the foregoing compounds can be used. Combinations of these compounds can also be used. In some embodiments the organic diamine is m-phenylenediamine, p-phenylenediamine, 4,4′-diaminodiphenyl sulfone , or a combination comprising one or more of the foregoing.

In some embodiments, diamine (8) is a meta-phenylene diamine (8a) or a para-phenylene diamine (8b)

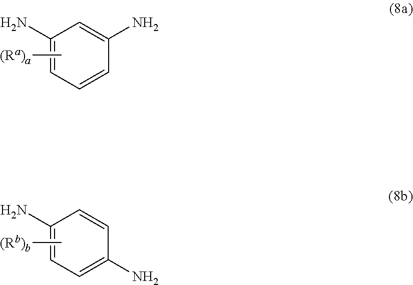

wherein R^{a} and R^{b} are each independently a halogen atom, nitro, cyano, C₂-C₂₀ aliphatic group, or C₂-C₄₀ aromatic group, and a and b are each independently 0 to 4. Examples include meta-phenylenediamine (mDA), para-phenylenediamine (pDA), 2,4-diaminotoluene, 2,6-diaminotoluene, 2-methyl-4,6-diethyl-1,3-phenylenediamine, 5-methyl-4,6-diethyl-1,3-phenylenediamine, and 1,3-diamino-4-isopropylbenzene. In some embodiments, diamine (8) is meta-phenylene diamine, para-phenylene diamine, 4,4′-diamino diphenyl sulfone, 4,4′-diaminodiphenyl sulfone, 3,4′-diaminodiphenyl sulfone, 3,3′-diaminodiphenyl sulfone, or a combination comprising at least one of the foregoing or a combination comprising at least one of the foregoing.

Imidization of substituted phthalic anhydride (7) and diamine (8) can be conducted in the absence or presence of a catalyst.

In general practice, a molar ratio of substituted phthalic anhydride (7) to diamine (8) of 1.98:1 to 2.2:1, specifically 1.98:1 to 2.1, or about 2:1 is used. A proper stoichiometric balance between substituted phthalic anhydride (7) and diamine (8) is maintained to prevent undesirable by-products that can limit the molecular weight of the polymer, or result in polymers with amine end groups. Accordingly, in some embodiments, imidization proceeds by adding diamine (8) to a mixture of substituted phthalic anhydride (7) and solvent to form a reaction mixture having a targeted initial molar ratio of substituted phthalic anhydride to diamine; heating the reaction mixture to a temperature of at least 100° C. (optionally in the presence of an imidization catalyst); analyzing the molar ratio of the heated reaction mixture to determine the actual initial molar ratio of substituted phthalic anhydride (7) to diamine (8); and, if necessary, adding substituted phthalic anhydride (7) or diamine (8) to the analyzed reaction mixture to adjust the molar ratio of substituted phthalic substituted phthalic anhydride (7) to diamine (8) to 1.98:1 to 2.2:1, preferably 2.0 to 2.1. Endcapping agents, such as mono-anhydrides or monoamines, or branching agents may also be employed in the reaction.

After imidization, the bis(phthalimide) (8) is polymerized by displacement, i.e., reaction with an alkali metal salt of a dihydroxy aromatic compound to provide the polyetherimide (1). In particular, the leaving group X of bis(phthalimide) (9)

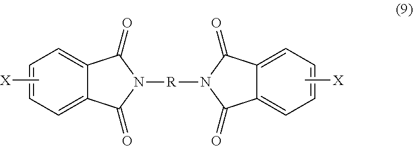

is displaced by reaction with an alkali metal salt of a dihydroxy aromatic compound of formula (10)

MO—Z—OM (10)

wherein M is an alkali metal and Z is as described in formula (1), to provide the polyetherimide of formula (1)

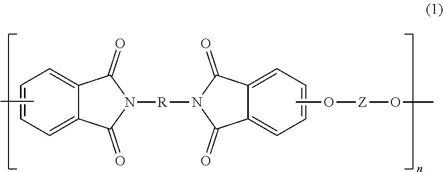

wherein n, R, and Z are as defined in formula (1).

Alkali metal M can each independently be any alkali metal, for example, lithium, sodium, potassium, and cesium, and can be the same as M² (discussed below). Thus alkali metal salt (10) can be lithium salts, sodium salts, potassium salts, cesium salts, or a combination comprising at least one of the foregoing. In some embodiments the metals are potassium or sodium. In some embodiments, M is sodium. The alkali metal salt (10) can be obtained by reaction of the metal hydroxide or carbonate with an aromatic dihydroxy compound of formula (4), specifically an aromatic C₆₋₂₄ monocyclic or polycyclic dihydroxy compound optionally substituted with 1 to 6 C₁₋₈ alkyl groups, 1 to 8 halogen atoms, or a combination thereof, for example, a bisphenol compound of formula (11)

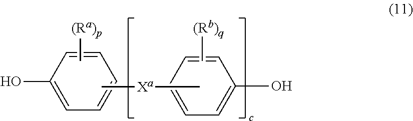

wherein R^{a}, R^{b}, and X^{a} are as described in formula (3). In some embodiments, the dihydroxy compound corresponding to formula (3a) can be used. The compound 2,2-bis(4-hydroxyphenyl) propane (“bisphenol A” or “BPA”) can be used.

The polymerization can be conducted in the presence of an alkali metal salt of a monohydroxy aromatic compound of formula (12)

M²O—Z² (12)

wherein M² is an alkali metal and Z² is a monohydroxy aromatic compound.

Alkali metal M² can be any alkali metal, for example, lithium, sodium, potassium, and cerium, and is generally the same as the alkali metal M. Thus alkali metal salt (12) is lithium salts, sodium salts, potassium salts, cesium salts, or a combination comprising at least one of the foregoing. In some embodiments, the metals are potassium or sodium. In some embodiments, M² is sodium. The alkali metal salt (12) can be obtained by reaction of the metal M² with aromatic C₆₋₂₄ monocyclic or polycyclic monohydroxy compound optionally substituted with 1 to 6 C₁₋₈ alkyl groups, 1 to 8 halogen atoms, or a combination thereof, for example, a monohydroxy aromatic compound formula (13)

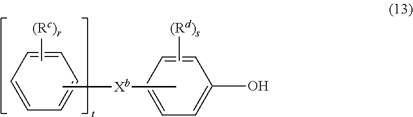

wherein R^{c} and R^{d} are each independently a halogen atom or a monovalent hydrocarbon group; r and s are each independently integers of 0 to 4; c is zero to 4; t is 0 or 1; when t is zero, X^{b} is hydrogen or a C₁₋₁₈ alkyl group; and when t is 1, X^{b} is a single bond, —O—, —S—, —S(O)—, —S(O)₂—, —C(O)—, or a C₁₋₁₈ organic bridging group. The C₁₋₁₈ organic bridging group can be cyclic or acyclic, aromatic or non-aromatic, and can further comprise heteroatoms such as halogens, oxygen, nitrogen, sulfur, silicon, or phosphorous. The C₁₋₁₈ organic bridging group can be disposed such that the C₆ arylene groups connected thereto are each connected to a common alkylidene carbon or to different carbons of the C₁₋₁₈ organic bridging group. In some embodiments, t is zero and X^{b} is hydrogen or a C₄₋₁₂ alkyl group or t is one and X^{b} is a single bond or a C₁₋₉ alkylene group.

In some embodiments, Z² is a group of formulas (13a)

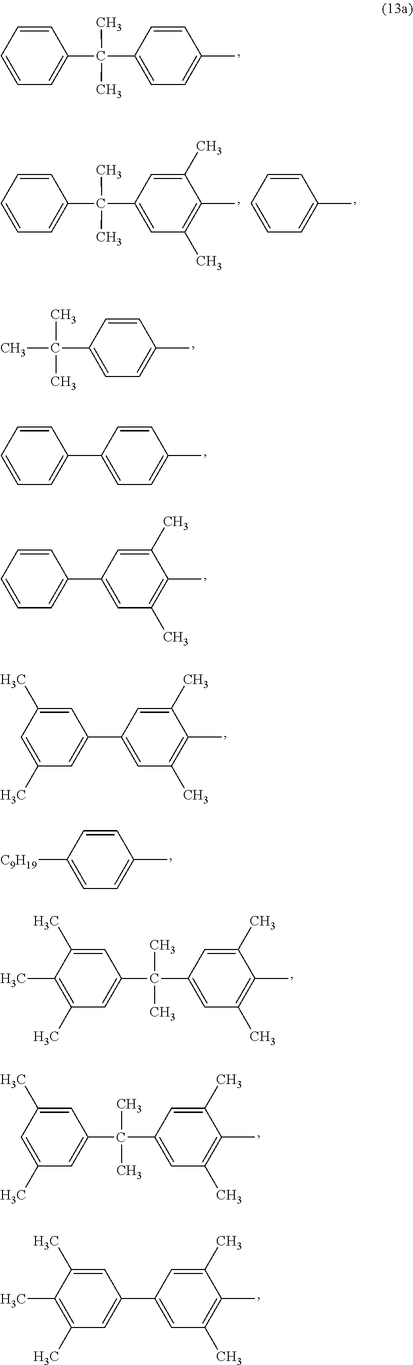

or a combination comprising at least one of the foregoing.

In some embodiments, Z and Z² are each independently a C₁₂₋₂₄ polycyclic hydrocarbyl moiety optionally substituted with 1 to 6 C₁₋₈ alkyl groups. In some embodiments, M and M² are each sodium. For example, in some embodiments, Z is a divalent group having formula

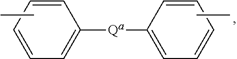

and Z² is a monovalent group having formula

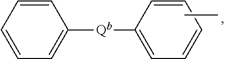

wherein Q^{a} and Q^{b} are each independently a single bond, —O—, —S—, —C(O), —SO₂, —SO—, —C_{y}H_{2y}— wherein y is an integer from 1 to 5, —(C₆H₁₀)_{z}— wherein z is an integer from 1 to 4; and a halogenated derivative thereof; and R is a divalent group having any one or more of the formulas (3) above.

Polymerization by reaction of bis(phthalimide) (9) with a combination of alkali metal salts (10) and (12) can be in the presence of phase transfer catalyst that is substantially stable under the reaction conditions used, in particular temperature. Exemplary phase transfer catalysts for polymerization include hexa(C₁₋₁₂ alkyl)guanidinium and α,ω-bis(penta(C₁₋₁₂ alkyl)guanidinium)alkane salts. Both types of salts can be referred to herein as “guanidinium salts.”

Polymerization is generally conducted in the presence of a relatively non-polar solvent, preferably with a boiling point above 100° C., specifically above 150° C., for example, o-dichlorobenzene, dichlorotoluene, 1,2,4-trichlorobenzene, diphenyl sulfone, sulfolane, a monoalkoxybenzene such as anisole, veratrole, diphenylether, or phenetole. Ortho-dichlorobenzene and anisole can be particularly mentioned.

Polymerization can be conducted at least 110° C., specifically 150° C. to 275° C., more specifically 175° C. to 225° C. At temperatures below 110° C., reaction rates may be too slow for economical operation. Atmospheric or super-atmospheric pressures can be used, for example, up to 5 atmospheres, to facilitate the use of high temperatures without causing solvent to be lost by evaporation.

In some embodiments, the combination of alkali metal salts (10) and (12) is added directly to the composition containing the bis(phthalimide) (9) in organic solvent. Water removal from the system can be accomplished in either batch, semi-continuous, or continuous processes, for example use of a distillation column in conjunction with one or more reactors. In some embodiments, a mixture of water and non-polar organic liquid distilling from a reactor is sent to a distillation column where water is taken off overhead and solvent is recycled back into the reactor at a rate to maintain or increase the desired solids concentration. Other methods for water removal include passing the condensed distillate through a drying bed for chemical or physical adsorption of water. The molar ratio of the bis(phthalimide) (9) to the alkali metal salt (10) can be 0.9:1 to 1.1:1.0.

### EXAMPLES

Materials used in the Examples are listed in Table 1. Amounts listed in the Examples are in weight percent (wt. %), based on the total weight of the identified composition.

TABLE 1

| Material | Chemical Description | Source |
|---|---|---|
| DDS | 4,4-diaminodiphenyl sulfone | Aldrich |
| mPD | meta-phenylene diamine | DuPont |
| 4-ClPA | 4-Chlorophthalic anhydride | SABIC |
| 3-ClPA | 3-Chlorophthalic anhydride | SABIC |
| PA | Phthalic anhydride | Aldrich |
| H₃PO₄ | Phosphoric Acid | Fischer |
| Na₂BPA | Disodium Bisphenol A | SABIC |
| oDCB | Ortho-dichlorobenzene | Fischer |
| HEGCl | Hexaethylguanidinium Chloride | SABIC |

### Comparative Example A

Purpose: To demonstrate a procedure for making ClPAMI which manifests a problem in ClPAMI production due to increased viscosity and extreme difficulty in stirring when the ClPAMI-ODCB slurry product reached a concentration of 20-22% solids.

A 3-necked 1 liter round-bottomed flask was equipped with a mechanical stirrer, a nitrogen inlet and a Dean-Stark trap and was charged with chlorophthalic anhydride (a 95/5 mixture of 4- and 3-isomers, 69.30 grams, 0.38 mol) and phthalic anhydride (0.57 gram, 3.8 mmol). The mixture was combined with o-dichlorobenzene (434.0 grams). The mixture was placed in a pre-heated oil bath and allowed to stir under a nitrogen blanket at 175° C. for 30 minutes. To this mixture was added m-phenylenediamine (20.67 grams, 0.19 mol) by means of a solid addition funnel over 30 minutes. An additional 20 grams of ODCB was used to rinse the funnel and ensure quantitative transfer of mPD. At this point the concentration was approximately 15% solids. The temperature was increased to 180° C. and 47 grams of ODCB and 5-6 milliliters of water boiled off and were collected. The concentration of ClPAMI at this point was at approximately 16% solids. The mixture was stirred for 5 hours and a nitrogen sweep was applied to concentrate the slurry. After further removal of 80 grams of ODCB, it was observed that the slurry had stopped mixing and a mass of agglomerated ClPAMI had formed on the sides of the vessel, on the stir shaft and through-out the reaction mixture (at this stage the concentration was at approx. 20% solids). The nitrogen blanket was restored and the mixing was continued. After 1 hour, HEGCl (2.5 grams, 20% solution in ODCB) was added and stirred for another hour. No change in characteristics of the slurry was observed during this time. Analysis by HPLC indicated that residual starting materials (3-ClPA, 4-ClPA, PA and mPD) and intermediates (4MA, 3MA) were present and within specifications. Some large agglomerated particles were observed as the slurry was stirred. Increasing the concentration to 25% solids, rendered the slurry impossible to stir and mix. Large agglomerated masses of ClPAMI appeared. Lowering the temperature of the slurry to around 100° C. afforded a slurry that looked almost like a cake.

### Example 1

Purpose. To demonstrate the procedure in making low viscosity ClPAMI at 30% solids with polymer additive upfront in ClPAMI synthesis.

A 3-necked 1 liter round-bottomed flask was equipped with a mechanical stirrer, a nitrogen inlet and a Dean-Stark trap and was charged with ULTEM brand polyetherimide in o-dichlorobenzene solution (5.0 grams, 22% solids), chlorophthalic anhydride (typically a 95/5 mixture of 4- and 3-isomers, 69.30 grams, 0.38 mol) and phthalic anhydride (0.57 gram, 3.8 mmol). The mixture was combined with o-dichlorobenzene (434.0 grams). The mixture was placed in a pre-heated oil bath and allowed to stir under a nitrogen blanket at 175° C. for 30 minutes. To this mixture was added m-phenylenediamine (20.67 grams, 0.19 mol) by means of a solid addition funnel over 30 minutes. An additional 20 grams of o-dichlorobenzene was used to rinse the funnel and ensure quantitative transfer of mPD. At this point the concentration was approximately 15% solids. The temperature was increased to 180° C. and 47 grams of ODCB and 5-6 milliliters of water were boiled off and collected. The concentration at this point was at approximately 16% solids. The mixture was stirred for 5 hours and a nitrogen sweep was applied to concentrate the slurry. After 2 hours (110 grams ODCB had been removed and concentration was now at approx. 21% solids), HEGCl (2.5 grams of a 20 wt % solution of HEGCl in ODCB) were added and stirred for another hour and during this time an additional 120 grams of ODCB was removed by means of a nitrogen sweep. The concentration of the slurry was at 30% solids and the slurry appeared to be stirring and remained free of agglomerated ClPAMI. The nitrogen blanket was restored and the mixing was continued. Analysis by HPLC indicated that residual starting materials (3-ClPA, 4-ClPA, PA and mPD) and intermediates (4MA, 3MA) were present and within specifications. This ClPAMI slurry was polymerized with BPA disodium salt. A polyetherimide polymer was obtained after workup with expected properties. The amount of polyetherimide polymer added to the ClPAMI reaction mixture was 1 wt % with respect to the amount of polymer produced in the reaction of the ClPAMI with the BPA disodium salt.

### Example 2

Purpose. To demonstrate the procedure in making low viscosity ClPAMI at 30% solids with polymer additive added later, after residuals specs have been achieved.

A 3-necked 1 liter round-bottomed flask was equipped with a mechanical stirrer, a nitrogen inlet and a Dean-Stark trap and was charged with chlorophthalic anhydride (typically a 95/5 mixture of 4- and 3-isomers, 69.30 grams, 0.38 mol) and phthalic anhydride (0.57 gram, 3.8 mmol). The mixture was combined with o-dichlorobenzene (434.0 grams). The mixture was placed in a pre-heated oil bath and allowed to stir under a nitrogen blanket at 175° C. for 30 minutes. To this mixture was added m-phenylenediamine (20.67 grams, 0.19 mol) by means of a solid addition funnel over 30 minutes. An additional 20 grams of ODCB was used to rinse through transfer surfaces and ensure quantitative transfer of mPD. At this point the concentration was approximately 15% solids. The temperature was increased to 180° C. and 47 grams of ODCB and 5-6 milliliters of water were boiled off and collected. The concentration at this point was 17% solids. The mixture was stirred for 5 hours and a nitrogen sweep was applied to concentrate the slurry. After further removal of 80 grams of ODCB, the slurry had stopped mixing and agglomerated ClPAMI had formed on the sides of the vessel, on the stir shaft and throughout the mixture (at this stage the concentration was at 20% solids). HEGCl (2.5 grams of a 20 wt % solution of HEGCl in ODCB) was added and the slurry stirred for an additional hour. The slurry remained thick and contained large agglomerated particles after 1 hour of stirring. Commercial ULTEM polyetherimide in ODCB solution (5.0 grams of a 22 wt % solution of ULTEM in ODCB) was added and the stirring was continued. After 1 to 2 minutes, the agglomerated material had disappeared and the slurry could be mixed without any significant deposit or ClPAMI agglomerates forming. ODCB was further removed from the slurry and concentration reached 30% solids. Some agglomerated material was observed in the slurry, but without loss of stirring. Analysis by HPLC indicated that residual starting materials (3-ClPA, 4-ClPA, PA, and mPD) and intermediates (4MA, 3MA) were within specifications. The amount of polymer added to the ClPAMI reaction mixture was 1 wt % with respect to the amount of polymer produced in the reaction of the ClPAMI with the BPA disodium salt.

### Example 3

Purpose. To demonstrate the procedure in making low viscosity ClPAMI at 30% solids with polymer additive upfront. The ClPAMI was polymerized with BPA-Na using a double slurry recipe.

The ClPAMI was made exactly as outlined in Example 1. The polymerization followed a double slurry recipe procedure. A 3-necked 1 liter round-bottomed flask was equipped with a mechanical stirrer, a nitrogen inlet and a Dean-Stark trap and was charged with commercial ULTEM polyetherimide in o-dichlorobenzene solution (5.0 grams of a 22 wt % solution of ULTEM in ODCB), chlorophthalic anhydride (typically a 95/5 mixture of 4- and 3-isomers, 69.30 grams, 0.38 mol) and phthalic anhydride (0.57 gram, 3.8 mmol). The mixture was combined with o-dichlorobenzene (434.0 grams). The mixture was placed in a pre-heated oil bath and allowed to stir under a nitrogen blanket at 175° C. for 30 minutes. To this mixture was added m-phenylenediamine (20.67 grams, 0.19 mol) by means of a solid addition funnel over 30 minutes. An additional 20 grams of o-dichlorobenzene was used to rinse the funnel and ensure quantitative transfer of mPD. At this point the concentration was approximately 15% solids. The temperature was increased to 180° C. and 47 grams of ODCB and 5-6 milliliters of water were boiled off and collected. The concentration at this point was at approximately 16% solids. The mixture was stirred for 5 hours and a nitrogen sweep was applied to concentrate the slurry. The ClPAMI reaction mixture was analyzed and found to be on stoichiometry (i.e., the ratio of ClPA to mPD was found to be 0.15 mol % excess ClPA). The amount of residual monoamine was 0.6 mol %. BPANa₂/ODCB (20 wt % solids) was then added to the ClPAMI reaction mixture. The ClPAMI/BPANa2 slurry product was concentrated to 30% solids (polymerization concentration) before HEGCl was added. Previous double slurry methodology, without adding ULTEM polymer to the reaction mixture, produced up to about 20 wt % solids before HEGCl was added.

### Comparative Example B

Purpose. To demonstrate a procedure for preparing DDS ClPAMI which exhibits a problem due to increased viscosity and extreme difficulty in stirring when the DDS ClPAMI-ODCB slurry product reached a concentration of 20-22% solids.

A 3-necked 250-mL Euro-type flask equipped with a mechanical stirrer, a nitrogen inlet and a Dean-Stark trap was charged with DDS (11.90 grams, 48 mmol) and 150 grams of ODCB. The temperature of the oil bath was raised to 185° C. and the mixture was stirred until all the solids had dissolved. Using a solid addition funnel, 3-chlorophthalic anhydride (17.38 grams, 95.2 mmol) was added slowly. Midway through the addition of the 3-ClPA, phthalic anhydride (0.135 gram, 0.91 mmol) was added. After the addition was complete, 25 grams of ODCB was used to rinse the sides of the addition funnel. A total of 78 grams of ODCB distillate containing droplets of water was removed over the course of 5 hours. The stoichiometry was adjusted by adding either DDS or 3-ClPA. After stoichiometric adjustment, 0.625 gram of HEGCl (20% in ODCB) was added. The mixture was stirred for 24 hours. The slurry appeared very thick and almost impossible to mix. The maximum concentration with loss of agitation was 22% before stirring was almost impossible.

### Example 4

Purpose. To demonstrate a process that overcame the thickness problem by adding 1% (with respect to the total amount of polymer produced in a subsequent reaction) of the desired end product polymer dissolved in ODCB prior to charging of DDS, 3-ClPA and PA, enabling the production of 30% solids DDS ClPAMI without loss of agitation.

A 3-necked 250-mL Euro-type flask equipped with a mechanical stirrer, a nitrogen inlet and a Dean-Stark trap was charged with 0.365 gram of polymer pellets (made from 3,3-DDS ClPAMI and BPANa₂) and 140 grams of ODCB. The temperature of the oil bath was raised to 185° C. and the mixture was stirred until the pellets had dissolved (1 hour). DDS (11.90 grams, 48 mmol) was charged directly to the reactor. The funnel was rinsed with 15 grams of ODCB and the mixture was stirred until all the solids had dissolved (1 hour). Using a solid addition funnel, a mixture of phthalic anhydride (0.135 gram, 0.91 mmol) and 3-chlorophthalic anhydride (17.38 grams, 95.2 mmol) was added. The funnel was rinsed with 15 grams of ODCB. A total of 78 grams of ODCB with droplets of water was removed over the course of 5 hours. The stoichiometry was adjusted by adding either DDS or 3-ClPA. The mixture was stirred for 24 hours at 185° C. The mixture was 22% solids. The slurry appeared to be very thin. Further distillation of ODCB until 30% solids was possible without loss of agitation. The slurry remained thin and could be stirred. Upon standing and cooling, the DDS ClPAMI settled and separated from a layer of ODCB.

### Comparative Example C

Demonstrates a known process.

The chloro-displacement reaction involves heating a mixture of DDS, ODCB, 3-ClPA, and SPP such that the formulation of the DDS ClPAMI was 25% solids. The mixture is then heated under pressure to produce the DDS ClPAMI. The pressure in the reactor was raised to 25 psig (pounds per square inch, gauge) to allow the temperature of the mixture to rise to 230° C., above the 180° C. boiling point of ODCB at 1 atm. The viscosity of the reaction mixture is less at 230° C. than at 180° C., due to the fact that the solubility of the product is greater at 230° C. than 180° C. In addition, the imidization rate was also improved at 230° C. over 180° C. After a 2-hour hold, the batch was sampled, analyzed, and additional correction of either DDS or 3-ClPA was added to bring the DDS ClPAMI on-stoichiometry. Once on-stoichiometry, the batch was held for 10 hours to finish the imidization. After these steps, the DDS ClPAMI slurry was diluted to 20% solids by addition of ODCB, pressure was released to 1 atm and the temperature dropped to 180° C. The batch was then azeotropically dried so that the overheads condensate was below 20 ppm water content. At 20% solids, the DDS ClPAMI slurry was viscous. If concentration was higher than 20% solid, the slurry was very sticky and stirring became impossible or very difficult.

### Comparative Example D

Purpose. To demonstrate a prior method to make BPA disodium slurry in ODCB.

In a 500 milliliter 3-neck Euro-type flask equipped a magnetic stirrer, nitrogen inlet and a means to connect to a bubbler was charged with BPA (57.07 g, 0.25 mol). The flask was sealed and the contents subjected to a repeated vacuum-purge cycle (5×). Using an addition funnel, 300 milliliter of degassed deionized water containing 0.50 mol of sodium hydroxide was added with constant stirring, the temperature was ramped to 90° C. until all the solids had dissolved.

In a separate 1-liter, 3-necked round-bottomed flask equipped a Dean-Stark trap and condenser attached to a bubbler and an inlet for nitrogen was charged with degassed ODCB (400 g). The contents of the flask were heated to 160° C. with the use of an external oil bath, under a blanket of nitrogen. Using a peristaltic pump, the aqueous BPA salt solution (maintained at 80 to 85° C.) was added drop wise to the stirring hot ODCB. Simultaneously during this step, ODCB-water azeotrope was collected in the Dean-Stark trap. The ODCB was continuously returned to the pot while the water phase was separated and collected. The BPANa₂ precipitated from the ODCB to afford a slurry. After all the aqueous BPA salt had been added, the temperature of the slurry was raised to 180° C. and ODCB was distilled until the distillate became clear and the salt concentration was 15% solids.

Using this process, the salt slurry appeared white but with a significant amount of large agglomerated salt particles. The slurry was placed inside a nitrogen box and transferred to a jar where it was subjected to a tissue homogenizer for at least 5 minutes. The slurry looked “grainy” and some particles were not broken up and settled at the bottom.

### Example 5

Purpose. To demonstrate a novel method to make BPA disodium slurry in ODCB using ODCB spiked with polyetherimide.

In a 500 milliliter 3-neck Euro-type flask equipped a magnetic stirrer, nitrogen inlet and a means to connect to a bubbler was charged with BPA (57.07 g, 0.25 mol). The flask was sealed and the contents subjected to a repeated vacuum-purge cycle (5×). Using an addition funnel, 300 milliliter of degassed deionized water was added and with constant stirring, the temperature was ramped to 90° C. until all the solids had dissolved.

In a separate 1-liter 3-neck round-bottomed flask equipped a Dean-Stark trap and condenser attached to a bubbler and an inlet for nitrogen was charged with degassed ODCB (400 g) and ULTEM polymer ODCB solution (2.0 g of a 22 wt % solution of ULTEM polymer in ODCB). The contents of the flask were heated to 190° C. under a blanket of nitrogen. Using a peristaltic pump, the aqueous BPA salt solution was added drop wise to the stirring hot ODCB. Simultaneously during this step, ODCB-water azeotrope was collected in the Dean-Stark trap. The ODCB was continuously returned to the pot while the water phase was separated and collected. After all the aqueous BPA salt had been added, the temperature was raised and ODCB was distilled until the distillate becomes clear and the salt concentration was 15% solids.

Using this process, the salt slurry appeared white with significantly less of the large agglomerated salt particles. It was observed that during the drying stage, agglomerates and lumps were broken up to finer particles of BPA salt. The slurry was placed inside a nitrogen box and transferred to a jar. The slurry had a uniform free flowing consistency and further grinding by tissue homogenizer was not needed. A sample of the slurry was squeezed between gloved fingers and the solids had the feel of powdery nature. It was easily crushed and did not feel “grainy”.

| | Temp | | | | |
|---|---|---|---|---|---|
| Comp. Ex A | | 175° C. 15% | 180° C. 16% | 20% (chunky) | 25% @ 100° C. cake-like |
| Ex. 1 (early | | 175° C. 15% at | 180° C. 16% | 21% add HEGCl | Concentrated to 30%, |
| polymer add) | | mPD add | | | stirring and |
| | | | | | agglomerate-free |
| Ex. 2 (late | | 15% at | 17% | 20% add HEGCl | Concentrated to 30%, |
| polymer add) | | mPD add | | (chunky) then add | stirrable with some |
| | | | | polymer | agglomerates |
| Ex. 3 (early | | | | | 30% solids |
| polymer/double | | | | | ClPAMI + BPANa₂ |
| slurry) | | | | | before catalyst |
| Comp. Ex. B | 185° C. | | | | 22% thick, stirrer |
| DDS | | | | | stopped |
| Ex. 4 | 185° C. | | | 22% thin slurry | Conc. to 30% |
| Comp. Ex. C | 190° C. | BPA + NaOH | | | Slurry grainy, particle |
| BPANa₂ | | dropped into | | | agglomerates don't |
| salt slurry | | ODCB | | | separate |
| Ex. 5 BPANa₂ | 190° C. | BPA + NaOH | | | Slurry particles are |
| Salt slurry with | | dropped into | | | finer size and flowable |
| polymer | | ODCB containing | | | |
| | | polymer | | | |

This disclosure is further illustrated by the following Embodiments, which are not intended to limit the claims.

Embodiment 1. A method for the manufacture of a polyetherimide composition, the method comprising imidizing a substituted phthalic anhydride having the formula

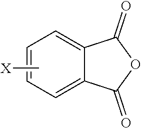

with an organic diamine having the formula H₂N—R—NH₂, in a solvent, at a temperature from 140° C. to 220° C., and a pressure from 0 psig to 100 psig, in the presence or absence of a phase transfer catalyst, to form a bis(phthalimide) having the formula

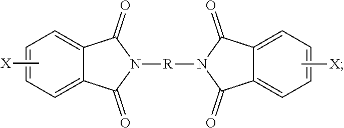

and polymerizing the bis(phthalimide) with an alkali metal salt of a dihydroxy aromatic compound having the formula MO—Z—OM to form the polyetherimide comprising the structural units having the formula

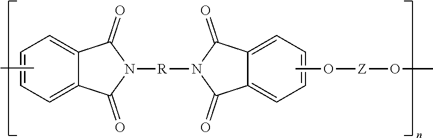

wherein in the foregoing formulae X is fluoro, chloro, bromo, iodo, nitro, or a combination comprising at least one of the foregoing; and each R is independently a C₆₋₂₀ aromatic hydrocarbon group, a straight or branched chain C₂₋₂₀ alkylene group, or a C₃₋₈ cycloalkylene group; M is an alkali metal; each Z is independently an aromatic C₆₋₂₄ monocyclic or polycyclic moiety; and n is an integer greater than 1; wherein a polyetherimide polymer is added before, during, or after the imidization reaction, to produce a bis(phthalimide) composition having a percent solids content of 18% to 30%, which bis(phthalimide) composition has a viscosity of less than 4000 cP at a shear rate of less than 30 sec⁻¹ and at a temperature from 140° C. to 180° C. as measured in a spindle viscometer.

Embodiment 2. The method of embodiment 1, wherein the added polyetherimide polymer is polyetherimide polymer liquid, polyetherimide polymer pellets, or polyetherimide polymer pre-devolatization solution.

Embodiment 3. The method of any one or more of embodiments 1-2, wherein the solids content of bis(phthalimide) is 25% to 30%.

Embodiment 4. The method of any one or more of embodiments 1-3, wherein hexaethylguanidinium chloride is present during the imidizing, the polymerizing, or both.

Embodiment 5. A method for the manufacture of a bis(phthalimide) comprising imidizing a substituted phthalic anhydride having the formula

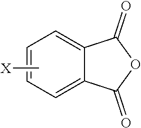

with an organic diamine having the formula H₂N—R—NH₂, in a solvent, at a temperature from 140° C. to 220° C., and a pressure from 0 psig to 100 psig, in the presence or absence of a phase transfer catalyst, to form a bis(phthalimide) having the formula

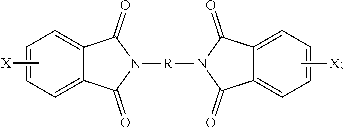

wherein a polyetherimide polymer is added either before, during, or after the imidization reaction, to produce a bis(phthalimide) composition having a percent solids content of 18% to 30%, which bis(phthalimide) composition has a viscosity of less than 4000 cP at a shear rate of less than 30 sec⁻¹ and at a temperature from 140° C. to 180° C. as measured in a spindle viscometer.

Embodiment 6. The method of embodiment 5, wherein the polyetherimide is polyetherimide polymer liquid, polyetherimide polymer pellets, or polyetherimide polymer pre-devolatization solution.

Embodiment 7. The method of any one or more of embodiments 5-6, wherein the solids content of bis(phthalimide) is 25% to 30%.

Embodiment 8. The method of any one or more of embodiments 5-7, wherein hexaethylguanidinium chloride is present during the imidizing.

Embodiment 9. A method for reducing the viscosity of a slurry of 10 to 30% solids ClPAMI in ortho-dichlorobenzene, by adding a polyetherimide polymer in an amount from 0.5 to 5 weight percent, or from 1 to 3 weight percent, or from 1 to 2 weight percent, each based on the weight of the ClPAMI solids, to provide improved mixing properties with a reduction in the slurry viscosity of 20% to 60%, at temperatures of 140° C. to 220° C., over a range of shear rates of 50 sec⁻¹ to 5 sec⁻¹ as measured in a spindle viscometer.

Embodiment 10. A method for producing ClPAMI at 30% solids in ortho-dichlorobenzene comprising adding polyetherimide polymer in an amount of from 0.5 to 5 weight percent, or from 1 to 3 weight percent, or from 1 to 2 weight percent, each based on the weight of the ClPAMI solids to produce a 30% solids ClPAMI slurry in ortho-dichlorobenzene, having a viscosity of less than 4000 cP at a shear rate of less than 30 sec⁻¹ at 165° C. as measured in a spindle viscometer.

Embodiment 11. A bis(phthalimide) product, preferably a ClPAMI product, having a solids content of 30% and a viscosity of less than 4000 cP at a shear rate of less than 30 sec⁻¹ at 165° C. as measured in a spindle viscometer.

Embodiment 12. A bis(phthalimide) product preferably a ClPAMI product, having a solids content of 30% and a viscosity of less than 4000 cP at a shear rate of less than 15 sec⁻¹ at 165° C. as measured in a spindle viscometer, preferably wherein the bis(phthalimide) is a bis(halophthalimide), more preferably ClPAMI.

Embodiment 13. A bis(phthalimide) product, preferably a ClPAMI product, having a solids content of 30% and a viscosity of less than 4000 cP at a shear rate of less than 10 sec⁻¹ at 165° C. as measured in a spindle viscometer, preferably wherein the bis(phthalimide) is a bis(halophthalimide), more preferably ClPAMI.

Embodiment 14. A method for preparing a bisphenol A disodium slurry in ortho-dichlorobenzene comprising charging ortho-dichlorobenzene and a polyetherimide polymer to a reactor, maintaining the reactor temperature above 100° C., and then gradually adding aqueous bisphenol A disodium slurry to the reactor.

Embodiment 15. The method of embodiment 14, wherein the aqueous bisphenol disodium slurry is added dropwise.

Embodiment 16. The method of any one or more of the preceding claims, wherein X is chloro, M is sodium, each Z is independently a divalent bisphenol A moiety, each R is independently m-phenylene, p-phenylene, bis(4,4′-phenylene)sulfone, bis(3,4′ phenylene) sulfone, or bis(3,3′-phenylene)sulfone.

The compositions and methods can alternatively comprise, consist of, or consist essentially of, any appropriate components or steps herein disclosed. The compositions or methods can additionally, or alternatively, be formulated so as to be devoid, or substantially free, of any components, materials, ingredients, adjuvants or species used in the prior art compositions or that are otherwise not necessary to the achievement of the function or objectives of the present claims.

All ranges disclosed herein are inclusive of the endpoints, and the endpoints are independently combinable with each other. “Combination” is inclusive of blends, mixtures, alloys, reaction products, and the like. The terms “a” and “an” and “the” do not denote a limitation of quantity, and are to be construed to cover both the singular and the plural, unless otherwise indicated herein or clearly contradicted by context. “Or” means “and/or” unless clearly stated otherwise. Reference throughout the specification to “some embodiments”, “an embodiment”, and so forth, means that a particular element described in connection with the embodiment is included in at least one embodiment described herein, and may or may not be present in other embodiments. In addition, it is to be understood that the described elements may be combined in any suitable manner in the various embodiments.

As used herein, the term “hydrocarbyl” includes groups containing carbon, hydrogen, and optionally one or more heteroatoms (e.g., 1, 2, 3, or 4 atoms such as halogen, O, N, S, P, or Si). “Alkyl” means a branched or straight chain, saturated, monovalent hydrocarbon group, e.g., methyl, ethyl, i-propyl, and n-butyl. “Alkylene” means a straight or branched chain, saturated, divalent hydrocarbon group (e.g., methylene (—CH₂—) or propylene (—(CH₂)₃—)). “Alkenyl” and “alkenylene” mean a monovalent or divalent, respectively, straight or branched chain hydrocarbon group having at least one carbon-carbon double bond (e.g., ethenyl (—HC═CH₂) or propenylene (—HC(CH₃)═CH₂—). “Alkynyl” means a straight or branched chain, monovalent hydrocarbon group having at least one carbon-carbon triple bond (e.g., ethynyl). “Alkoxy” means an alkyl group linked via an oxygen (i.e., alkyl-O—), for example methoxy, ethoxy, and sec-butyloxy. “Cycloalkyl” and “cycloalkylene” mean a monovalent and divalent cyclic hydrocarbon group, respectively, of the formula —CₙH₂ₙ₋ₓ and —CₙH₂ₙ₋₂ₓ— wherein x is the number of cyclization(s). “Aryl” means a monovalent, monocyclic, or polycyclic aromatic group (e.g., phenyl or naphthyl). “Arylene” means a divalent, monocyclic or polycyclic aromatic group (e.g., phenylene or naphthylene). The prefix “halo” means a group or compound including one more halogen (F, Cl, Br, or I) substituents, which can be the same or different. The prefix “hetero” means a group or compound that includes at least one ring member that is a heteroatom (e.g., 1, 2, or 3 heteroatoms, wherein each heteroatom is independently N, O, S, or P.

Unless substituents are otherwise specifically indicated, each of the foregoing groups can be unsubstituted or substituted, provided that the substitution does not significantly adversely affect synthesis, stability, or use of the compound. “Substituted” means that the compound, group, or atom is substituted with at least one (e.g., 1, 2, 3, or 4) substituents instead of hydrogen, where each substituent is independently nitro (—NO₂), cyano (—CN), hydroxy (—OH), halogen, thiol (—SH), thiocyano (—SCN), C₁₋₆ alkyl, C₂₋₆ alkenyl, C₂₋₆ alkynyl, C₁₋₆ haloalkyl, C₁₋₉ alkoxy, C₁₋₆ haloalkoxy, C₃₋₁₂ cycloalkyl, C₅₋₁₈ cycloalkenyl, C₆₋₁₂ aryl, C₇₋₁₃ arylalkylene (e.g, benzyl), C₇₋₁₂ alkylarylene (e.g, toluyl), C₄₋₁₂ heterocycloalkyl, C₃₋₁₂ heteroaryl, C₁₋₆ alkyl sulfonyl (—S(═O)₂-alkyl), C₆₋₁₂ arylsulfonyl (—S(═O)₂-aryl), or tosyl (CH₃C₆H₄SO₂—), provided that the substituted atom's normal valence is not exceeded, and that the substitution does not significantly adversely affect the manufacture, stability, or desired property of the compound. When a compound is substituted, the indicated number of carbon atoms is the total number of carbon atoms in the compound or group, including those of any substituents.

All references are incorporated herein by reference.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

1. A method for the manufacture of a polyetherimide composition, the method comprising:
imidizing a substituted phthalic anhydride having the formula 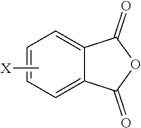
with an organic diamine having the formula H₂N—R—NH₂, in a solvent, at a temperature from 140° C. to 220° C., and a pressure from 0 psig to 100 psig, in the presence or absence of a phase transfer catalyst, to form a bis(phthalimide) having the formula 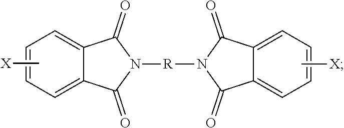
and
polymerizing the bis(phthalimide) with an alkali metal salt of a dihydroxy aromatic compound having the formula
MO—Z—OM
to form the polyetherimide comprising the structural units having the formula 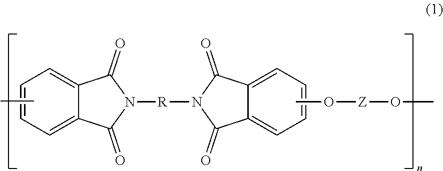
wherein in the foregoing formulae
X is fluoro, chloro, bromo, iodo, nitro, or a combination comprising at least one of the foregoing; and
each R is independently a C₆₋₂₀ aromatic hydrocarbon group, a straight or branched chain C₂₋₂₀ alkylene group, or a C₃₋₈ cycloalkylene group;
M is an alkali metal;
each Z is independently an aromatic C₆₋₂₄ monocyclic or polycyclic moiety; and
n is an integer greater than 1;
wherein a polyetherimide polymer is added before, during, or after the imidization reaction, to produce a bis(phthalimide) composition having a percent solids content of 18% to 30%, which bis(phthalimide) composition has a viscosity of less than 4000 cP at a shear rate of less than 30 sec⁻¹ and at a temperature from 140° C. to 180° C. as measured in a spindle viscometer.

2. The method of claim 1, wherein the added polyetherimide polymer is polyetherimide polymer liquid, polyetherimide polymer pellets, or polyetherimide polymer pre-devolatization solution.

3. The method of claim 1, wherein the solids content of bis(phthalimide) is 25% to 30%.

4. The method of claim 1, wherein hexaethylguanidinium chloride is present during the imidizing, the polymerizing, or both.

5. A method for the manufacture of a bis(phthalimide) comprising imidizing a substituted phthalic anhydride having the formula 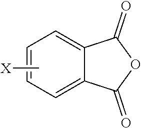
with an organic diamine having the formula H₂N—R—NH₂, in a solvent, at a temperature from 140° C. to 220° C., and a pressure from 0 psig to 100 psig, in the presence or absence of a phase transfer catalyst,
to form a bis(phthalimide) having the formula 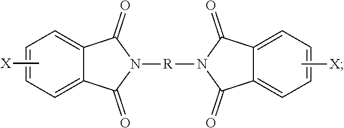
wherein a polyetherimide polymer is added either before, during, or after the imidization reaction, to produce a bis(phthalimide) composition having a percent solids content of 18% to 30%, which bis(phthalimide) composition has a viscosity of less than 4000 cP at a shear rate of less than 30 sec⁻¹ and at a temperature from 140° C. to 180° C. as measured in a spindle viscometer.

6. The method of claim 5, wherein the polyetherimide is polyetherimide polymer liquid, polyetherimide polymer pellets, or polyetherimide polymer pre-devolatization solution.

7. The method of claim 5, wherein the solids content of bis(phthalimide) is 25% to 30%.

8. The method of claim 5, wherein hexaethylguanidinium chloride is present during the imidizing.

9. A method for reducing the viscosity of a slurry of 10 to 30% solids ClPAMI in ortho-dichlorobenzene, by adding a polyetherimide polymer in an amount from 0.5 to 5 weight percent, based on the weight of the ClPAMI solids, to provide improved mixing properties with a reduction in the slurry viscosity of 20% to 60%, at temperatures of 140° C. to 220° C., over a range of shear rates of 50 sec⁻¹ to 5 sec⁻¹ as measured in a spindle viscometer.

10. The method of claim 9, wherein the slurry has ClPAMI at 30% solids and the polyetherimide polymer is added in an amount of from 1 to 3 weight percent, based on the weight of the ClPAMI solids to produce a 30% solids ClPAMI slurry in ortho-dichlorobenzene, having a viscosity of less than 4000 cP at a shear rate of less than 30 sec⁻¹ at 165° C. as measured in a spindle viscometer.

11. A bis(phthalimide) product, having a solids content of 30% and a viscosity of less than 4000 cP at a shear rate of less than 30 sec⁻¹ at 165° C. as measured in a spindle viscometer.

12. The bis(phthalimide) product of claim 11, wherein the bis(phthalimide) product is a ClPAMI product.

13. The bis(phthalimide) product of claim 11, having a solids content of 30% and a viscosity of less than 4000 cP at a shear rate of less than 10 sec⁻¹ at 165° C. as measured in a spindle viscometer.

14. A method for preparing a bisphenol A disodium slurry in ortho-dichlorobenzene comprising charging ortho-dichlorobenzene and a polyetherimide polymer to a reactor, maintaining the reactor temperature above 100° C., and then gradually adding aqueous bisphenol A disodium slurry to the reactor.

15. The method of claim 14, wherein the aqueous bisphenol disodium slurry is added dropwise.

16. The method of claim 1, wherein
X is chloro,
M is sodium,
each Z is independently a divalent bisphenol A moiety,
each R is independently m-phenylene, p-phenylene, bis(4,4′-phenylene)sulfone, bis(3,4′-phenylene)sulfone, or bis(3,3′-phenylene)sulfone.
